(19) United States
(12) Patent Application Publication
(10) Pub. No.: US 2008/0045611 A1
(43) Pub. Date: Feb. 21, 2008

(54) Methods for the therapeutic use of a carbonic anhydrase inhibitor for reducing spinal cord edema

(76) Inventor: Milan Radojicic, Santa Rosa, CA (US)

Correspondence Address: Milan Radojicic, 4145 Shadow Lane, Apt. 435, Santa Rosa, CA, 95405, US

(21) Appl. No.: 11/840,206

(22) Filed: Aug. 16, 2007

Related U.S. Application Data
(60) Provisional application No. 60/822,640, filed on Aug. 17, 2006.

(51) Int. Cl. A61K 45/00

(52) U.S. Cl. 514/789

(57) ABSTRACT

This invention relates to methods of treating mammals at risk or affected by spinal cord edema by administering a carbonic anhydrase inhibitor that crosses the blood-spinal cord barrier. Among the conditions treated are, but not limited to, spinal cord injury, spinal cord tumors and vascular malformations, spinal infections, transverse myelitis, multiple sclerosis, syringomyelia and the pre-syrinx state.

### CROSS-REFERENCE TO RELATED APPLICATIONS

This application claims the benefit of provisional patent application Ser. No. 60/822,640, filed Aug. 17, 2006 by the present inventor.

### FEDERALLY SPONSORED RESEARCH

Not applicable.

### SEQUENCE LISTING OR PROGRAM

Not applicable.

### BACKGROUND OF THE INVENTION

1. Field of the Invention

The field of the present invention relates to methods of reducing spinal cord edema in mammals using a carbonic anhydrase inhibitor that crosses the blood-spinal cord barrier. In particular, the invention relates to methods of administering therapeutically effective amounts of a carbonic anhydrase inhibitor that crosses the blood-spinal cord barrier to a mammal affected or at risk for spinal cord edema for the purposes of producing a therapeutic affect.

2. Prior Art

By the end of the next decade, 300,000 people will be living with chronic spinal cord injury in the US alone. Advances in medical and rehabilitative care have improved survival rates for these individuals, but many experience clinical decline even years after the initial injury. Clinical decline is often accompanied by a slow and progressive cavitation of the central spinal cord, known as post-traumatic syringomyelia. The pathogenesis of this disease remains poorly understood, but may be related to spinal cord edema. Beyond spinal cord injury, other spinal diseases can result in spinal cord edema. Such diseases include spinal tumors and vascular malformation, spinal infections, multiple sclerosis, transverse myelitis, and non-traumatic syringomyelia.

Spinal cord edema may result from alterations extracellular fluid processing as well as alterations in cerebrospinal fluid (CSF) dynamics following spinal cord injury. This is thought to play a role in the initiation and propagation of post-traumatic syringomyelic cysts and cavities.

Following spinal cord injury and disease, normal CSF dynamics may be distorted by a number of possible mechanisms, including subarachnoid CSF outflow obstructions, changes in compliance of the subarachnoid space, or elevated intraspinal pressures. Altered CSF dynamics are believed to result in localized spinal cord edema, known as the presyrinx state that subsequently gives rise to central canal dilation and/or the formation of intraspinal glial-lined parenchymal cysts.

Increasingly, spinal cord edema is being recognized as potent force in the morbidity and mortality of spinal cord injury and disease. Post-traumatic steroids have been utilized with mixed results and frequent untoward effects. However, until this Applicant's invention, no safe and cost-effective method or device has been applied to cerebrospinal fluid diversion

The use of a carbonic anhydrase inhibitor in brain edema has been subject of patents, for example, see Rodriguez U.S. Pat. No. 5,755,237; Rodriguez U.S. Pat. No. 5,944,021. However, until this Applicant's invention, no application of the carbonic anhydrase inhibitor has been utilized in spinal cord edema.

### DETAILED DESCRIPTION OF THE INVENTION

This invention relates to methods of treating mammals at risk or affected by spinal cord edema by administering a carbonic anhydrase inhibitor that crosses the blood-spinal cord barrier. Among the conditions treated are, but not limited to, spinal cord injury, spinal cord tumors and vascular malformations, spinal infections, transverse myelitis, multiple sclerosis, syringomyelia and the pre-syrinx state.

In the preferred embodiment, the administering step comprises intravenous administration of the carbonic anhydrase inhibitor that crosses the blood-spinal cord barrier in patients at risk for spinal cord edema or in patients recently diagnosed with the disorder. Topical administration allows a therapeutically active amount of the cyclosporine component to be delivered locally without substantial systemic absorption and side effects.

In another embodiment, the administering step comprises the mucosal administration of the carbonic anhydrase inhibitor that crosses the blood-spinal cord barrier.

Carbonic anhydrase inhibitors are a class of pharmaceuticals act as an enzyme inhibitor upon carbonic anhydrase. It can act as a mild diuretic, but is thought to reduce central nervous system edema and pressure by reducing cerebrospinal fluid production.

Carbonic anhydrase inhibitors have the advantage over stronger diuretics, such as Mannitol and Lasix, of avoiding large scale fluid shifts and hemodynamic instability. Carbonic anhydrase inhibitors have the advantage over steroids of avoiding infections, tissue breakdown and related untoward effects.

Any suitable carbonic anhydrase inhibitor that crosses the blood brain barrier effective in the present methods may be used.

### EXAMPLE 1

A male patient, age 24, suffers a fractured thoracic vertebrae and spinal cord injury. Immediately after laminectomy and surgical fixation, he is noted to have a decline in his neurological exam, with loss of sensory and motor functions in his hands. An emergent MRI demonstrates a significant amount of spinal cord edema ascending toward the cervical cord. An intravenous dose of 500 mg of acetazolamide in sterile saline is administered. Within a short period, he regains sensory and motor functions in his upper extremities.

### EXAMPLE 2

A female patient, age 54, undergoes radiation therapy for a malignant lesion of her neck. Post-radiation, she develops quadriplegia, cranial nerve palsies and is emergently intubated. A STAT MRI demonstrates significant edema at the level of the cervical spinal cord. A continuous infusion of a carbonic anhydrase inhibitor at 1000 mg/day is begun, with resolution of her deficits and extubation by day 3.

While the above description contains many specificities, these should not be construed as limitations on the scope of the invention, but as exemplifications of the presently preferred embodiments thereof. Many other ramifications and variations are possible within the teaching of the invention. Additionally, any combination of the above examples may be possible.

Thus the scope of the invention should be determined by the appended claims and their legal equivalents, rather than the examples given.

1. A method for reducing spinal cord edema in a mammal comprising administering of a carbonic anhydrase inhibitor that passes through the blood-spinal cord barrier to said mammal in an amount sufficient to reduce spinal cord edema.

2. The method of claim 1, wherein the administration of the carbonic anhydrase inhibitor compound is accomplished by a method selected from the group consisting of mucosal, injection, intramuscular injection, intravenous injection and implantation infusion device.

3. A method according to claim 2, wherein the administration of the carbonic anhydrase inhibitor compound includes a dosage of said carbonic anhydrase inhibitor of about 250 mg to 1000 mg per day.
